(19) United States
(12) Patent Application Publication
(10) Pub. No.: US 2026/0166338 A1
(43) Pub. Date: Jun. 18, 2026

(54) REAL TIME DOSIMETRY SYSTEMS AND METHODS

(71) Applicant: WearableDose, Inc, Bowie, MD (US)

(72) Inventor: John B. Sanwo, Bowie, MD (US)

(73) Assignee: WearableDose, Inc, Bowie, MD (US)

(21) Appl. No.: 19/346,559

(22) Filed: Sep. 30, 2025

Related U.S. Application Data
(60) Provisional application No. 63/701,513, filed on Sep. 30, 2024.

(51) Int. Cl. A61N 5/10

(52) U.S. Cl. CPC A61N 5/1031; A61N 2005/1074; A61N 2005/1087

(57) ABSTRACT

A method for dynamic treatment planning may comprise receiving a radiation treatment plan for a patient. The radiation treatment plan may comprise a plurality of sessions to be provided to the patient. A real time dose sensor may be arranged proximate to a skin surface of the patient. The real time dose sensor may provide realtime radiation awareness through continuous monitoring capabilities across healthcare, defense, and infrastructure applications. The sensor may enable radiation detection through electronic polymer materials configured to exhibit conductivity changes upon exposure. A measurement of actual radiation dose delivered to the target volume may be received from the sensor. Modification may support precision cancer therapy through adaptive dose optimization. The platform may extend beyond medical applications to encompass radiation monitoring in military operations, nuclear facility surveillance, and environmental protection systems.

### PRIORITY DOCUMENTS

Under provisions of 35 U.S.C. § 119(e), the Applicant claims the benefit of U.S. Provisional Application No. 63/701,513, filed Sep. 30, 2024, which is incorporated herein by reference in its entirety. It is intended that each of the referenced applications may be applicable to the concepts and embodiments disclosed herein, even if such concepts and embodiments are disclosed in the referenced applications with different limitations and configurations and described using different examples and terminology.

### TECHNICAL FIELD

The present disclosure relates to systems and methods for real-time dosimetry in radiation therapy. More particularly, the disclosure relates to dynamic treatment planning systems that may utilize wearable electronic polymer sensors for real-time monitoring and adjustment of radiation doses during cancer treatment.

### BACKGROUND OF THE INVENTION

The following discussion of related art is provided to assist the reader in understanding the advantages of the present disclosure. This discussion may not constitute an admission that such related art is prior art to the present disclosure.

Radiation therapy may be utilized as a treatment modality for various types of cancer, including breast cancer, brain tumors, and other malignancies. External beam radiation therapy devices, such as linear accelerators, gamma ray therapy systems, and proton therapy machines, may deliver prescribed radiation doses to target volumes within patients.

Current radiation therapy approaches may face several challenges. Traditional static treatment planning methods may not account for real-time variations in dose delivery. Patient movement during treatment may affect dosing accuracy. Collateral damage to healthy tissues surrounding target volumes may occur due to imprecise dose delivery. Side effects from radiation therapy may impact patient quality of life.

Existing dosimetry solutions may have limitations. Optically stimulated luminescence (OSL) dosimeters may provide delayed feedback rather than real-time monitoring. Traditional diode detectors may lack personalization and real-time adjustment capabilities. Immobilization techniques and optical monitoring systems may not detect all patient movements with required precision.

The recent recall of widely used OSLD dosimeters may underscore the need for more reliable and advanced solutions in radiation therapy. Studies may indicate that up to 95% of patients experience some level of skin reaction, and around 50% may suffer from significant fatigue and other complications due to inaccurate dosing and insufficient real-time monitoring.

There may be a need for systems and methods that provide comprehensive radiation monitoring and dynamic treatment adaptation across multiple therapeutic modalities to enhance patient outcomes and minimize adverse effects.

### BRIEF SUMMARY OF THE INVENTION

The present disclosure may provide systems and methods for real-time dosimetry that address limitations of conventional radiation therapy approaches. The disclosure may not be limited to the specific embodiments described herein, and various modifications and alternative configurations may be implemented without departing from the scope of the disclosure.

According to one aspect of the disclosure, a system for dynamic treatment planning may be provided. The system may include one or more wearable dose sensors configured to be positioned proximate to a skin surface of a patient. The system may include an external radiation therapy device configured to deliver radiation doses to a target volume of the patient. The system may include a treatment planning engine configured to receive dosimetric data from the wearable dose sensors during radiation delivery and modify subsequent treatment sessions based on the received data. The system may further include radiation detection capabilities configured to operate across multiple domains including healthcare, defense, and infrastructure monitoring applications. The wearable dose sensors may be configured to detect radiation exposure in clinical environments, military operational areas, and civilian infrastructure settings. The treatment planning engine may be configured to process radiation data from diverse operational contexts while maintaining precision therapy capabilities.

According to another aspect of the disclosure, a method for dynamic treatment planning may be provided. The method may include receiving a radiation treatment plan comprising multiple sessions. The method may include arranging a real-time dose sensor proximate to a patient's skin surface. The method may include providing a first prescribed radiation dose during a first session while monitoring actual dose delivery through the sensor. The method may include modifying at least a second session based on measured actual radiation dose. The method may include providing a second prescribed radiation dose during the modified second session. The method may further include deploying the real-time dose sensor in non-clinical environments for radiation awareness applications. The method may include monitoring radiation exposure in defense operations, infrastructure protection, and emergency response scenarios. The method may include generating radiation awareness data for multi-domain applications while preserving clinical treatment planning functionality.

According to another aspect of the disclosure, a wearable dosimeter apparatus may be provided. The apparatus may include electronic polymer sensor elements configured to generate electrical signals in response to radiation exposure. The apparatus may include a flexible substrate configured to conform to patient skin surfaces. The apparatus may include signal processing circuitry configured to convert analog sensor signals to digital dosimetric data. The apparatus may include communication modules configured to transmit dosimetric data to treatment planning systems. The apparatus may further include multi-domain detection capabilities configured to operate in healthcare, defense, and infrastructure environments. The electronic polymer sensor elements may be configured to detect radiation across multiple operational contexts. The communication modules may be configured to interface with diverse monitoring systems including clinical networks, military communication systems, and civilian infrastructure platforms.

The systems and methods described herein may provide various advantages over conventional approaches. Real-time monitoring may enable dynamic adjustment of treatment parameters during therapy delivery. Wearable sensors may provide in-vivo dose measurements with high spatial resolution. AIML algorithms may optimize treatment plans based on real-time feedback. Patient safety may be enhanced through continuous monitoring and automatic adjustment capabilities. Multi-domain radiation detection may provide comprehensive awareness across healthcare, defense, and infrastructure applications. Cross-platform compatibility may enable deployment in diverse operational environments. Unified radiation monitoring may support precision therapy while extending to broader radiation awareness applications.

These and other aspects of the disclosure may be better understood with reference to the following description and drawings. The embodiments described herein may represent only some of many possible implementations of the disclosed subject matter. Features from different embodiments may be combined, and additional or alternative features may be incorporated without departing from the scope of the disclosure.

### BRIEF DESCRIPTION OF THE DRAWINGS

The accompanying drawings may be included to provide further understanding and may be incorporated in and constitute a part of this specification. The drawings may illustrate disclosed embodiments and together with the description may serve to explain principles of the disclosed subject matter. The drawings may not be drawn to scale, and relative dimensions may be modified for clarity of illustration.

FIG. 1 may illustrate a network architecture that may be used to implement dynamic treatment planning, according to some embodiments of the disclosure.

FIG. 2 may be a block diagram illustrating details of a system for dynamic treatment planning, according to some embodiments of the disclosure.

FIG. 3 may be a block diagram illustrating an exemplary computer system with which aspects of the subject technology may be implemented, according to some embodiments of the disclosure.

FIG. 4 may be a block diagram illustrating dynamic treatment planning for external radiation treatment, according to some embodiments of the disclosure.

FIG. 5 may illustrate a dosimetry solution for external beam machines, according to some embodiments of the disclosure.

FIG. 6 may illustrate a wearable patch that may be integrated into clothing, according to some embodiments of the disclosure.

FIG. 7 illustrates a real-time radiation awareness system for environmental monitoring and decision-making support.

In one or more implementations, not all of the depicted components in each figure may be required, and one or more implementations may include additional components not shown in a figure. Variations in the arrangement and type of the components may be made without departing from the scope of the subject disclosure. Additional components, different components, or fewer components may be utilized within the scope of the subject disclosure.

### DETAILED DESCRIPTION

The following detailed description may be provided to enable any person skilled in the art to make and use the disclosed subject matter. Various modifications to the disclosed embodiments may be apparent to those skilled in the art, and the generic principles defined herein may be applied to other embodiments and applications without departing from the scope of the disclosure. The present disclosure may not be limited to the embodiments shown, but may be accorded the widest scope consistent with the principles and features disclosed herein.

All references cited anywhere in this specification, including the Background and Detailed Description sections, may be incorporated by reference as if each had been individually incorporated. The disclosure may include various embodiments that may be implemented separately or in combination. Features described in connection with one embodiment may be applicable to other embodiments, and various combinations and subcombinations of features may be implemented without departing from the scope of the disclosure.

The present disclosure may relate to systems and methods for real-time dosimetry in radiation therapy applications. The disclosed systems may combine wearable electronic polymer sensors with dynamic treatment planning capabilities to provide precise, in-vivo monitoring and adjustment of radiation doses during cancer treatment.

### Real-Time Dosimetry System Architecture

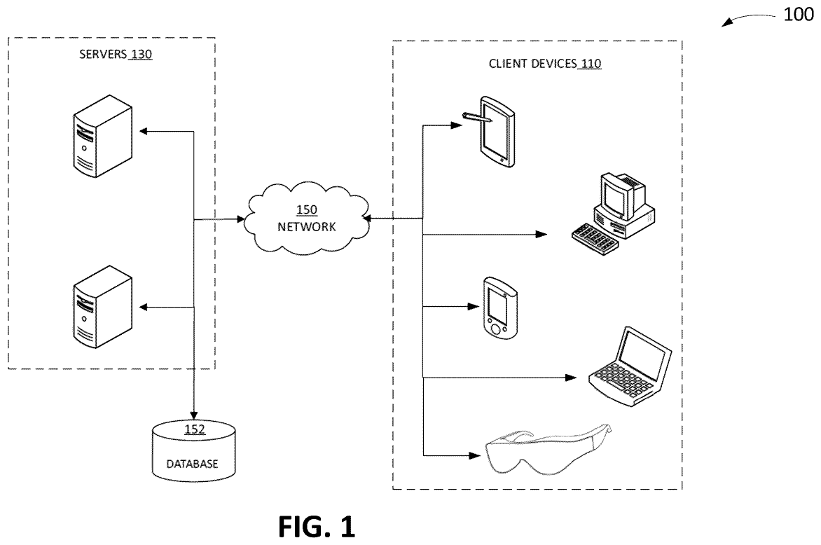

Referring to FIG. 1, a network architecture 100 may be used to implement dynamic treatment planning according to some embodiments. The network architecture 100 may include one or more client devices 110 and servers 130 that may be communicatively coupled via a network 150. The system may include at least one database 152 that may store data and files associated with the servers 130 and client devices 110.

The client devices 110 may collect data, video, images, and other information for upload to the servers 130 for storage in the database 152. The network 150 may include wired networks such as fiber optics, copper wire, and telephone lines. The network 150 may include wireless networks such as satellite networks, cellular networks, radiofrequency networks, Wi-Fi, and Bluetooth connections. The network 150 may include local area networks, wide area networks, the Internet, and other network topologies including bus networks, star networks, ring networks, and mesh networks.

The client devices 110 may include laptop computers, desktop computers, and mobile devices such as smart phones, tablets, televisions, wearable devices, head-mounted devices, and display devices. The servers 130 may be cloud servers or groups of cloud servers. The servers 130 may be implemented outside of cloud computing environments, including on-premises environments. The servers 130 may include rack-mounted computing devices, processing boards, switchboards, routers, and other network devices.

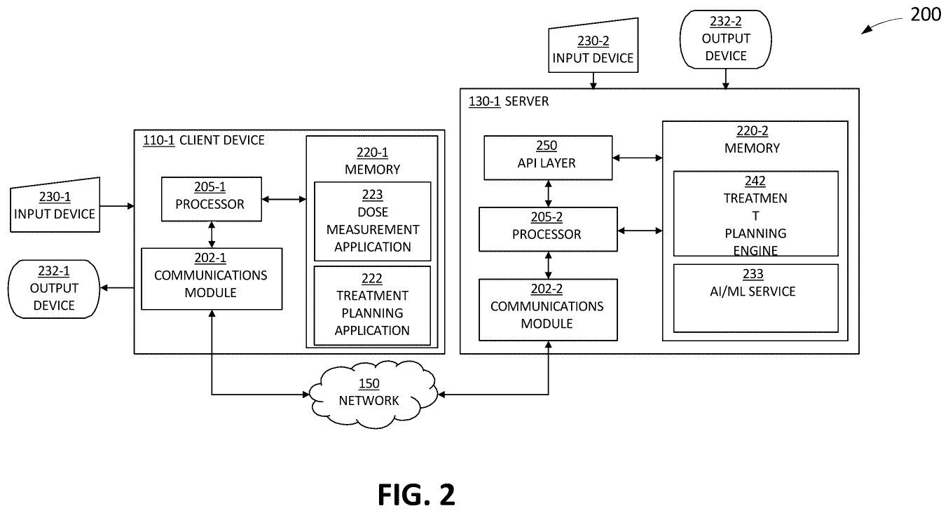

Referring to FIG. 2, a system 200 for dynamic treatment planning may include details of an exemplary client device 110-1 and an exemplary server 130-1 from the network architecture 100 of FIG. 1. The client device 110-1 and server 130-1 may be communicatively coupled over network 150 via respective communications modules 202-1 and 202-2.

The communications modules 202 may be configured to interface with network 150 to send and receive information such as requests, data, messages, and commands to other devices on the network 150. The communications modules 202 may be modems or Ethernet cards. The communications modules 202 may include radio hardware and software for wireless communications via electromagnetic radiation, radiofrequency, near field communications, Wi-Fi, and Bluetooth radio technology.

The client device 110-1 and server 130-1 may include processors 205-1 and 205-2 and memories 220-1 and 220-2, respectively. The processors 205 may be configured to execute instructions stored in memories 220 to cause the client device 110-1 and server 130-1 to perform methods and operations consistent with embodiments of the present disclosure.

The client device 110-1 and server 130-1 may be coupled to input devices 230-1 and 230-2, respectively. The input devices 230 may include a mouse, controller, keyboard, pointer, stylus, touchscreen, microphone, voice recognition software, joystick, virtual joystick, and touch-screen display. The input devices 230 may include cameras, microphones, sensors, touch sensors, acoustic sensors, and inertial motion units.

The client device 110-1 and server 130-1 may be coupled to output devices 232-1 and 232-2, respectively. The output devices 232 may include a screen, display, touchscreen display, speaker, and alarm. A user may interact with the client device 110-1 and server 130-1 via the input devices 230 and output devices 232.

The memory 220-1 may include a treatment planning application 222 configured to execute on client device 110-1 and couple with input device 230-1 and output device 232-1. The treatment planning application 222 may be downloaded by the user from server 130-1 and may be hosted by server 130-1. The treatment planning application 222 may include specific instructions which, when executed by processor 205-1, may cause operations to be performed consistent with embodiments of the present disclosure.

The memory 220-2 may include a treatment planning engine 242 configured to perform methods and operations consistent with embodiments of the present disclosure. The treatment planning engine 242 may share features and resources with the client device 110-1, including data, libraries, and applications. The user may access the treatment planning engine 242 through the treatment planning application 222.

The memory 220-1 may include a dose measurement application 223 configured to execute in client device 110-1. The dose measurement application 223 may communicate with AI/ML service 233 in memory 220-2 to provide dose measurement calculations. The dose measurement application 223 and treatment planning application 222 may communicate with the treatment planning engine 242 and AI/ML service 233 through API layer 250.

### Wearable Electronic Polymer Sensors

The disclosed systems may utilize wearable electronic polymer sensors for real-time radiation detection. The sensors may be based on electronic polymer dosimeter technology that may employ pi-conjugated polymer materials. These materials may exhibit conductivity changes upon radiation exposure, enabling real-time dose measurement.

In one or more embodiments, the wearable sensors may include a three-dimensional embedded electrode structure with electrically resistive materials. The sensors may be fabricated using pi-conjugated polymer materials that may generate electrical signals proportional to radiation dose. The sensors may provide spatial resolution capabilities down to 70 microns for precise dose mapping.

In one or more embodiments, the electronic polymer dosimeter patches may be positioned on patient skin surfaces proximate to treatment areas. The patches may be flexible and water-equivalent for tissue-equivalent measurements. The sensors may provide continuous in-vivo monitoring of radiation dose delivery during treatment sessions.

The wearable detector cup (WDC) may be configured with curved-surface detector arrays that may conform to treatment device geometry. Multi-sensor arrays may provide spatial dose distribution mapping. Integrated positioning systems may detect beam entrance locations for geometric verification.

### Dynamic Treatment Planning Engine

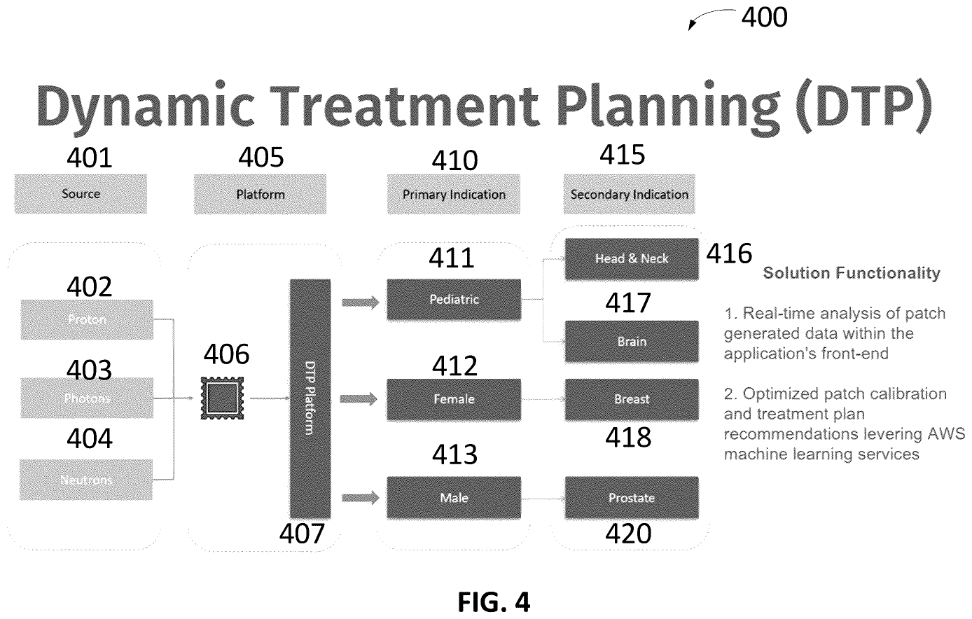

Referring to FIG. 4, dynamic treatment planning for external radiation treatment may be implemented according to some embodiments. The system may provide real-time analysis of data from wearable patches within a user interface of a treatment planning application. Optimized patch calibration and treatment plan recommendations may be provided by the application, leveraging machine learning and artificial intelligence services.

The treatment planning engine may receive initial radiation treatment plans with prescribed doses and durations. The engine may establish communication with wearable dose sensors during treatment delivery. Real-time dosimetric data from sensors may be processed during treatment sessions.

AI/ML algorithms may be executed to analyze dose delivery accuracy compared to planned parameters. Modified treatment parameters may be generated based on real-time feedback from sensors. Updated treatment plans may be transmitted to radiation therapy devices for implementation in subsequent sessions.

The system may archive treatment data for quality assurance and analysis purposes. Historical treatment data may be utilized to refine AI/ML algorithms and improve treatment planning accuracy. Patient-specific factors may be incorporated into dynamic treatment optimization.

Furthermore regarding FIGS. 4, 400 dynamic treatment planning for external radiation treatment may be implemented according to some embodiments. The dynamic treatment planning system may include a comprehensive platform architecture 400 that may integrate multiple treatment indications and solution functionality components. The system may include primary indication processing modules 410 and secondary indication 415 processing modules that may handle different treatment protocols.

The source 401 may incorporate protons 402, photons, and neutrons 404. The platform 405 may incorporate a processor 406 and DTP platform 407. The primary indication 410 may incorporate pediatric treatments 411, female treatments 412, and male treatments 413. The secondary indication 415 may incorporate treatment area modules for head and neck treatments 416, brain treatments 417, breast treatments 418, and prostate treatments 420. Each treatment area module may be configured to process specific anatomical considerations and dosimetric requirements for the respective treatment sites. The system may include clinic integration nodes that may facilitate communication between the platform and clinical treatment environments.

The solution functionality may require analysis of plan generation using synthetic and specialized test data through processing nodes. The system may incorporate optimized patch calibration environments that may be served via cloud inference leveraging machine learning services. The platform may include multiple site processing capabilities through distributed nodes that may enable multi-institutional treatment planning coordination.

The system may include normalization processing modules that may standardize treatment parameters across different treatment protocols. Additional processing nodes may handle specialized computational tasks for treatment optimization. The platform may incorporate microprocessor-based control units that may manage real-time treatment adjustments and parameter monitoring.

The dynamic treatment planning platform may include data integration capabilities through interface modules that may facilitate communication between wearable dosimeter sensors and treatment planning algorithms. The system may process real-time dosimetric feedback from electronic polymer sensors during treatment delivery sessions. Treatment plan modifications may be generated based on continuous monitoring of radiation dose distribution and patient positioning parameters.

The platform may utilize artificial intelligence algorithms to analyze dose delivery patterns and generate optimized treatment recommendations. Machine learning models may be trained on historical treatment data to improve prediction accuracy for patient-specific treatment responses. The system may provide automated alerts and recommendations when measured dose parameters deviate from planned treatment specifications.

### External Radiation Therapy Integration

The disclosed systems may be compatible with various external beam radiation therapy devices. Linear accelerators may be utilized for photon beam delivery with real-time dosimetric feedback. Gamma ray therapy devices such as GammaPod systems may be integrated with wearable detector cups for breast radiosurgery monitoring.

Proton therapy machines may be coupled with wearable sensors for precision proton beam delivery. The systems may provide dynamic dosing capabilities for proton therapy applications. Real-time monitoring may enable adjustment of proton beam parameters based on actual dose delivery measurements.

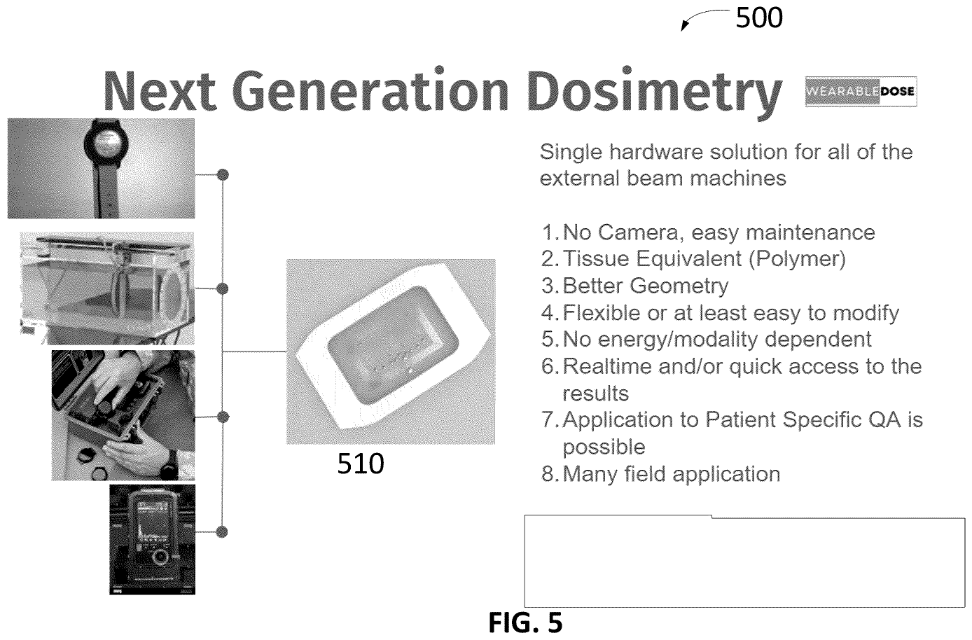

Referring to FIGS. 5, 500 a dosimetry solution for external beam machines may be provided according to some embodiments. The system may provide a single hardware solution for any external beam machine. Advantages may include no need for cameras, easy maintenance, tissue-equivalent polymer construction, improved geometry, flexibility, ease of modification, no energy or modality dependence, real-time access to results, application to patient-specific quality assurance, and various field applications.

The external radiation therapy devices may receive initial treatment plan parameters from the treatment planning engine. Radiation delivery may begin according to prescribed protocols. Real-time modification commands may be accepted from the treatment planning system. Beam parameters, timing, or positioning may be adjusted based on sensor feedback. Treatment delivery may be completed with optimized parameters.

Referring to FIG. 5, a dosimetry solution 500 for external beam machines may be provided according to some embodiments. The system 500 may provide a single hardware solution for any external beam machine. The dosimetry solution 500 may include a rectangular dosimeter device 510 that may be configured for tissue-equivalent radiation measurements. The device 510 may be fabricated from polymer materials that may provide homogeneous radiation response characteristics equivalent to human tissue.

The dosimetry solution 500 may include a clinical setup configuration that may enable direct contact positioning with treatment surfaces. The setup may allow for easy modification and flexible geometric arrangements to accommodate various treatment scenarios. The system may be configured to provide energy and angle independent measurement results across different radiation modalities.

The dosimetry solution 500 may incorporate electronic control equipment that may enable real-time measurement capabilities. The control equipment may include processing circuitry configured to provide modular and quick quality assurance measurements. The electronic systems may be designed to interface with existing treatment planning systems for seamless integration.

The dosimetry solution 500 may provide patient-specific quality assurance capabilities that may enable individualized treatment verification. The system may be configured to accommodate multiple field applications and various treatment geometries. The patient-specific features may allow for customized measurement protocols based on individual treatment requirements.

The dosimetry solution 500 may include versatile application capabilities that may enable use across many field applications and treatment scenarios. The system may be configured to provide consistent performance across different external beam radiation therapy devices. The versatile capabilities may include compatibility with linear accelerators, gamma ray therapy devices, and proton therapy systems.

The dosimetry solution 500 may provide advantages including elimination of camera requirements for easy maintenance operations. The system may utilize tissue-equivalent polymer construction that may provide homogeneous radiation response characteristics. The solution may enable direct contact measurements with flexible geometry configurations.

The dosimetry solution 500 may provide real-time measurement access and quick quality assurance capabilities. The system may be configured to deliver measurement results without dependency on specific energy levels or beam modalities. The real-time capabilities may enable immediate feedback during radiation delivery sessions.

The dosimetry solution 500 may include comprehensive field application support that may accommodate various treatment protocols and measurement scenarios. The system may be configured to provide consistent performance across different clinical environments and treatment devices. The field application support may include compatibility with multiple external beam radiation therapy platforms and treatment planning systems.

### Wearable Patch Integration

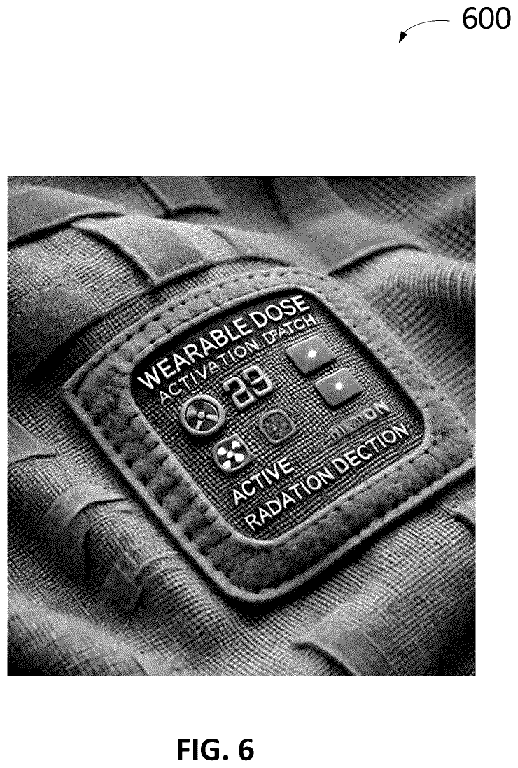

Referring to FIGS. 6, 600 a wearable patch may be integrated into clothing according to some embodiments. The patch may include a display showing dosimetric information such as radiation levels or exposure duration. Activation controls may enable users to turn the monitoring system on or off. Status indicators may show when active radiation detection is occurring.

The wearable patch 600 may incorporate electronic polymer sensors within a flexible substrate. The patch may be designed for comfortable wear during extended treatment sessions. Communication modules may transmit dosimetric data wirelessly to treatment planning systems. The patch may include user interface elements for status monitoring and control. Digital displays may show real-time dose measurements or accumulated exposure. Visual and audible alerts may notify users of dose threshold exceedances or system status changes.

The patch 600 may be configured with multiple sensor zones that may enable comprehensive radiation monitoring across different regions of the treatment area. Each sensor zone may contain multiple electronic polymer sensor elements arranged in a matrix configuration for enhanced spatial resolution. The patch may include a central processing unit that may coordinate data collection from all sensor zones and may perform preliminary signal processing before transmission to external systems.

The patch 600 may incorporate a flexible printed circuit board substrate that may provide electrical connections between sensor elements and control circuitry. The substrate may be fabricated from biocompatible materials that may conform to patient skin surfaces without causing irritation or discomfort during extended wear periods. The printed circuit board may include conductive traces that may route sensor signals to amplification and digitization circuits embedded within the patch structure.

The patch 600 may include environmental sensing capabilities that may monitor ambient conditions such as temperature, humidity, and pressure during treatment sessions. These environmental sensors may provide contextual data that may be used to compensate for external factors that could affect radiation measurements. The environmental data may be integrated with dosimetric measurements to provide comprehensive treatment monitoring information.

The patch 600 may be equipped with data storage capabilities that may enable local archiving of measurement data in case of communication interruptions with external systems. The storage system may include non-volatile memory elements that may retain measurement data even during power interruptions. The stored data may be automatically synchronized with treatment planning systems once communication is restored.

The patch 600 may include calibration capabilities that may enable field calibration using known radiation sources or reference measurements. The calibration system may adjust sensor response characteristics to account for manufacturing variations or environmental factors that could affect measurement accuracy. Calibration parameters may be stored in onboard memory and may be updated remotely through wireless communication interfaces.

The patch 600 may incorporate power management systems that may optimize battery life during extended treatment sessions. The power management circuitry may include sleep modes that may reduce power consumption during periods of inactivity while maintaining readiness for immediate measurement operations. Battery status information may be transmitted to treatment planning systems to provide advance warning of power depletion.

### Clinical Applications

The disclosed systems may be applied to various clinical radiation therapy scenarios. Breast cancer treatment may utilize GammaPod integration with wearable detector cups for real-time monitoring of stereotactic radiosurgery procedures. The system may provide objective and independent monitoring of treatment progress with automatic evaluation and triggering of action plans when problems are detected.

Pediatric brain tumor treatment may benefit from precision radiation monitoring with reduced need for anesthesia. Real-time dose adjustment may minimize damage to healthy brain tissues while ensuring adequate tumor coverage. Long-term monitoring capabilities may enable assessment of late effects and guide post-treatment care.

The systems may be applicable to various anatomical sites including head and neck, brain, breast, prostate, and other treatment locations. Primary indications may include pediatric, female, and male patient populations. Secondary indications may encompass multiple cancer types and treatment scenarios.

### Realtime Radiation Monitoring

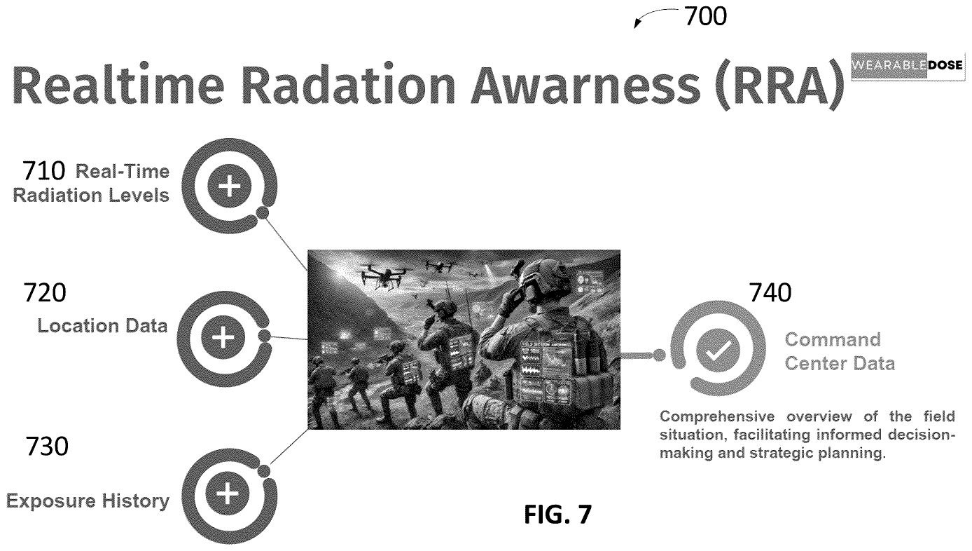

Referring to FIGS. 7, 700 may provide comprehensive real-time radiation awareness via real-time radiation monitoring capabilities through integrated data streams. The system 700 may include real-time radiation level monitoring 710 that may continuously track ambient radiation conditions across monitored areas. The real-time monitoring component 710 may utilize distributed sensor networks that may provide instantaneous feedback on radiation exposure levels throughout the operational environment.

The system 700 may incorporate location data processing 720 that may track the geographical positioning of radiation sources and monitoring equipment. The location data component 720 may integrate with global positioning systems to provide precise spatial coordinates for all monitored radiation events. The location tracking capabilities 720 may enable correlation between radiation measurements and specific geographical areas for enhanced situational awareness.

The real-time radiation awareness system 700 may include exposure history analysis 730 that may maintain comprehensive records of radiation exposure patterns over time. The exposure history component 730 may archive historical radiation data that may be used for trend analysis and predictive modeling. The historical data processing 730 may enable identification of radiation exposure patterns that may inform future treatment planning decisions.

The real-time radiation awareness system 700 may feature command center data integration 740 that may provide centralized coordination of all environmental monitoring functions. The command center component 740 may aggregate data from multiple monitoring sources to provide comprehensive situational awareness for decision-making personnel. The command center integration 740 may enable real-time visualization of radiation field conditions that may support strategic planning and operational response coordination.

The real-time radiation awareness system 700 may provide automated alert capabilities when radiation levels exceed predetermined thresholds. The system 700 may generate notifications that may be transmitted to appropriate personnel for immediate response. The monitoring platform 700 may enable remote access to radiation data through secure communication networks for distributed decision-making support.

The system 700 may integrate real-time radiation level data from component 710, location data from component 720, and exposure history information from component 730. Command center data from component 740 may provide situational awareness for radiation safety personnel. The system 700 may enable monitoring of radiation exposure in various environments including medical facilities, research laboratories, and emergency response scenarios.

The real-time radiation level monitoring 710 may employ wearable dose sensors that may be positioned on personnel operating in monitored environments. The wearable sensors may transmit radiation measurement data to the central processing system through wireless communication protocols. The monitoring component 710 may provide continuous dose rate measurements that may be processed for immediate alert generation.

The location data processing 720 may receive positioning information from GPS-enabled radiation detection devices. The component 720 may correlate radiation measurements with specific geographical coordinates to create spatial radiation maps. The location processing capabilities may enable tracking of radiation source movement and contamination spread patterns.

The exposure history analysis 730 may maintain databases of cumulative radiation exposure data for individual personnel and monitored areas. The component 730 may analyze temporal trends in radiation exposure to identify patterns that may indicate equipment malfunction or environmental changes. The historical analysis may support long-term health monitoring and safety protocol development.

The command center data integration 740 may receive data streams from all monitoring components to provide unified operational oversight. The integration component 740 may process multiple data sources to generate comprehensive radiation field assessments. The command center capabilities may enable coordinated response to radiation incidents across multiple operational areas.

### Computer System Implementation

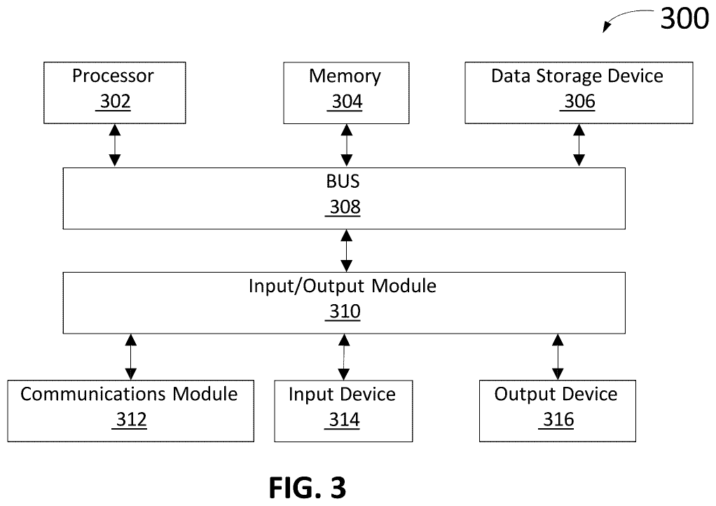

Referring to FIG. 3, an exemplary computer system 300 may be utilized to implement aspects of the subject technology according to some embodiments. The computer system 300 may be implemented using hardware or combinations of software and hardware in dedicated servers, integrated entities, or distributed systems.

The computer system 300 may include a bus 308 for communicating information and a processor 302 coupled with bus 308 for processing information. The processor 302 may be a general-purpose microprocessor, microcontroller, Digital Signal Processor, Application Specific Integrated Circuit, Field Programmable Gate Array, Programmable Logic Device, controller, state machine, gated logic, or discrete hardware components.

The computer system 300 may include memory 304 such as Random Access Memory, flash memory, Read-Only Memory, Programmable Read-Only Memory, Erasable PROM, registers, hard disk, removable disk, CD-ROM, DVD, or other suitable storage device coupled to bus 308 for storing information and instructions to be executed by processor 302.

Instructions may be stored in memory 304 and implemented in computer program products encoded on computer-readable media for execution by the computer system 300. The instructions may be implemented in various programming languages including data-oriented languages, system languages, architectural languages, and application languages.

The computer system 300 may include a data storage device 306 such as a magnetic disk or optical disk coupled to bus 308 for storing information and instructions. The computer system 300 may be coupled via input/output module 310 to various devices including communications module 312, input device 314, and output device 316.

### Method Implementations

The disclosed systems may implement various methods for dynamic treatment planning and real-time dosimetry. A method for dynamic treatment planning may include receiving a radiation treatment plan for a patient comprising multiple sessions to be provided to the patient. A real-time dose sensor may be arranged proximate to a skin surface of the patient.

During a first session of the radiation treatment plan, a first prescribed radiation dose may be provided to the patient using an external radiation therapy device. The dose may be delivered to a target volume of the patient during a first prescribed duration. The target volume may include the skin surface of the patient where the real-time dose sensor is arranged.

A measurement of actual radiation dose delivered to the target volume may be received from the sensor during the first session. At least a second session of the radiation treatment plan may be modified based on the measurement of actual radiation, resulting in a modified second session.

During the modified second session of the radiation treatment plan, a second prescribed radiation dose may be provided to the patient using the external radiation therapy device. The dose may be delivered to a target volume of the patient during a second prescribed duration. The target volume may include the skin surface of the patient where the real-time dose sensor is arranged.

The method may include various modifications and enhancements. Modifying the second session may include replacing an initial prescribed radiation dose with the second prescribed radiation dose. Modifying the second session may include replacing an initial prescribed radiation duration with the second prescribed radiation duration.

Multiple real-time dose sensors may be utilized to provide directional information about radiation treatment delivery. A first real-time dose sensor and a second real-time dose sensor may be arranged proximate to the skin surface of the patient. Modifying the second session may be further based on a measurement of the direction of the radiation treatment during the first session. The measurement of the direction of the radiation treatment may be based on measurements from the first real-time dose sensor and the second real-time dose sensor.

The external radiation therapy device may be a gamma ray therapy device, proton therapy device, linear accelerator, or other external beam radiation therapy system. The method may be applied to various cancer treatment scenarios including breast cancer, brain tumors, and other malignancies.

### Applications and Embodiments

The military system may provide cumulative radiation exposure tracking for individual personnel across multiple missions and deployments. The system may maintain historical exposure records that may be used for medical monitoring and safety protocol development. Personnel radiation exposure data may be archived for long-term health assessment and regulatory compliance purposes.

The drone-based detection platforms may include autonomous flight capabilities that may enable systematic radiation surveillance of large operational areas. The drones may follow predetermined flight patterns or respond automatically to specific radiation detection events by deploying to investigation areas. The drone systems may provide extended monitoring duration through battery management and automated recharging capabilities.

The military radiation detection system may include secure communication protocols that may protect sensitive operational data during transmission. The communication networks may utilize encryption and authentication mechanisms to prevent unauthorized access to radiation monitoring information. The secure data transmission may enable coordination between multiple operational units while maintaining operational security requirements.

The integrated monitoring platform may provide real-time coordination between patch-based personnel monitoring and drone-based area surveillance. The system may correlate individual exposure data with area contamination patterns to provide comprehensive radiation situational awareness. Command personnel may access unified displays that may show both individual personnel exposure levels and area radiation conditions simultaneously.

The responsiveness to radiation events may include automated deployment of additional monitoring resources when initial detection thresholds are exceeded. The system may trigger coordinated response protocols that may include personnel evacuation, medical assessment, and contamination containment procedures. The rapid response capabilities may minimize radiation exposure through immediate notification and protective action implementation.

### System Advantages and Benefits

The disclosed systems may provide various advantages over conventional radiation therapy approaches. Real-time monitoring may enable immediate detection and correction of dose delivery errors. Dynamic treatment planning may optimize therapy effectiveness while minimizing side effects.

Wearable sensors may provide continuous in-vivo dose measurements without requiring invasive procedures. Patient comfort may be improved through reduced need for immobilization devices or anesthesia. Treatment precision may be enhanced through high-resolution spatial dose mapping.

AI/ML integration may enable personalized treatment optimization based on individual patient responses. Historical data analysis may improve treatment planning accuracy over time. Quality assurance may be enhanced through comprehensive treatment monitoring and documentation.

The systems may reduce treatment session time by up to 60% and treatment duration by up to 40% compared to conventional approaches. Patient safety may be improved through real-time monitoring and automatic adjustment capabilities. Side effects may be minimized through precise dose delivery and real-time feedback control.

### Technical Advantages and Benefits

The disclosed systems may provide significant technical advantages through the integration of electronic polymer dosimeter technology with dynamic treatment planning capabilities. The electronic polymer dosimeter patches may utilize pi-conjugated polymer materials that may exhibit conductivity changes proportional to radiation exposure levels. These materials may provide superior signal-to-noise ratios compared to conventional dosimetric approaches through their direct electrical response to ionizing radiation.

The three-dimensional embedded electrode structure within the wearable patches may enable spatial resolution capabilities of approximately 70 microns. This enhanced spatial resolution may provide precise dose mapping capabilities that may exceed the performance of traditional dosimetric systems. The electrode structure may be fabricated using conductive traces that may route sensor signals through amplification and digitization circuits embedded within the flexible substrate.

The tissue-equivalent properties of the polymer materials may ensure accurate dose measurements that may correlate directly with patient tissue response to radiation exposure. The water-equivalent characteristics of the sensor materials may eliminate the need for complex correction factors that may be required with conventional dosimetric approaches. The flexible substrate construction may conform to patient skin surfaces without causing discomfort during extended treatment sessions.

Real-time data processing capabilities may enable immediate detection and correction of dose delivery variations during treatment sessions. The continuous monitoring approach may provide instantaneous feedback to treatment planning systems that may adjust beam parameters based on actual measured dose delivery. This dynamic adjustment capability may reduce treatment delivery errors that may occur with static treatment planning approaches.

The AI/ML integration within the treatment planning engine may analyze historical treatment data to optimize future treatment protocols. Machine learning algorithms may identify patterns in dose delivery accuracy that may improve treatment planning precision over time. The predictive capabilities may enable personalized treatment optimization based on individual patient response characteristics.

The system may provide compatibility with multiple external beam radiation therapy devices including linear accelerators, gamma ray therapy systems, and proton therapy machines. This universal compatibility may eliminate the need for device-specific dosimetric solutions that may be required with conventional monitoring approaches. The modular design may enable rapid deployment across different treatment platforms without extensive reconfiguration.

Environmental sensing capabilities within the wearable patches may monitor ambient conditions that may affect radiation measurements. Temperature, humidity, and pressure sensors may provide contextual data that may be used to compensate for external factors. The environmental compensation algorithms may ensure measurement accuracy across varying clinical conditions.

Power management systems within the patches may optimize battery life during extended treatment sessions. Sleep mode capabilities may reduce power consumption during periods of inactivity while maintaining readiness for immediate measurement operations. The power optimization may enable continuous monitoring throughout multi-session treatment protocols.

Data storage capabilities may enable local archiving of measurement data in case of communication interruptions with external systems. Non-volatile memory elements may retain measurement data even during power interruptions. The stored data may be automatically synchronized with treatment planning systems once communication is restored.

Calibration capabilities may enable field calibration using known radiation sources or reference measurements. The calibration system may adjust sensor response characteristics to account for manufacturing variations or environmental factors. Calibration parameters may be stored in onboard memory and may be updated remotely through wireless communication interfaces.

The wireless communication modules may transmit dosimetric data to treatment planning systems without physical connections that may interfere with treatment delivery. The communication protocols may ensure secure data transmission while maintaining low latency for real-time applications. Multiple communication pathways may provide redundancy to prevent data loss during critical treatment phases.

Quality assurance capabilities may be enhanced through comprehensive treatment monitoring and documentation. The system may provide objective verification of dose delivery accuracy compared to planned parameters. Automated alerts may notify personnel when measured parameters deviate from acceptable ranges.

The disclosed approach may reduce treatment session time by enabling more efficient dose delivery protocols. Real-time feedback may eliminate the need for conservative safety margins that may be required with conventional approaches. Treatment duration may be reduced through optimized dose delivery patterns based on continuous monitoring feedback.

Patient safety may be improved through immediate detection of dose delivery anomalies. The system may provide early warning of potential treatment errors before significant dose deviations occur. Automatic adjustment capabilities may correct minor variations without interrupting treatment delivery.

Clinical workflow integration may be simplified through standardized interfaces with existing treatment planning systems. The platform may accommodate various treatment protocols without requiring extensive staff retraining. User interface elements may provide intuitive access to treatment monitoring and adjustment capabilities.

The manufacturing processes for the wearable sensors may involve advanced polymer processing techniques that may enable precise control of material properties. The fabrication methods may include solution casting, spin coating, or printing technologies that may deposit sensor materials onto flexible substrates. The electrode structures may be formed using photolithographic patterning or conductive ink printing processes.

The sensor calibration procedures may utilize standardized radiation sources that may provide known dose rates for reference measurements. The calibration algorithms may account for temperature dependencies, humidity effects, and aging characteristics of the polymer materials. The calibration data may be stored in non-volatile memory within each sensor patch for field reference during operation.

The communication protocols between sensors and treatment planning systems may utilize wireless standards such as Bluetooth Low Energy or Wi-Fi for data transmission. The data packets may include sensor identification, timestamp information, dose measurements, and quality indicators. The transmission protocols may incorporate error correction and encryption capabilities to ensure data integrity and patient privacy.

The battery management systems within the wearable patches may monitor power consumption and may provide low-battery warnings to clinical personnel. The power optimization algorithms may adjust sampling rates and transmission frequencies based on treatment requirements and remaining battery capacity. The charging systems may utilize inductive coupling or USB connections for battery replenishment between treatment sessions.

The sensor arrays within each patch may be arranged in grid patterns that may provide spatial resolution capabilities for dose mapping. The individual sensor elements may be interconnected through conductive traces that may route signals to central processing units. The array configurations may be customizable based on treatment area requirements and anatomical considerations.

The signal amplification circuits may utilize low-noise operational amplifiers that may enhance sensor signal quality. The amplification stages may include programmable gain settings that may accommodate different radiation dose ranges. The analog-to-digital conversion systems may provide high-resolution digitization of sensor signals for accurate dose calculations.

The data processing algorithms may apply filtering techniques that may remove electrical noise and interference from sensor measurements. The signal processing may include baseline correction, drift compensation, and temperature normalization functions. The processed data may be formatted for transmission to treatment planning systems through standardized interfaces.

The user interface elements on the wearable patches may include LED indicators that may provide visual status information. The indicator patterns may convey operational status, battery level, and measurement activity to clinical personnel. The visual feedback systems may utilize color coding to communicate different operational states and alert conditions.

The environmental monitoring capabilities may include temperature sensors that may track ambient conditions during treatment sessions. The humidity sensors may detect moisture levels that could affect sensor performance. The pressure sensors may monitor contact conditions between the patch and patient skin surface.

The data logging capabilities may record all sensor measurements with precise timestamps for post-treatment analysis. The logged data may include raw sensor readings, processed dose values, environmental conditions, and system status information. The data storage systems may utilize circular buffer techniques to manage memory usage during extended monitoring periods.

The patch adhesion systems may utilize biocompatible adhesives that may maintain secure contact with patient skin throughout treatment sessions. The adhesive materials may be designed to minimize skin irritation while providing reliable mechanical attachment. The removal procedures may be designed to prevent skin damage when patches are detached after treatment completion.

The sterilization procedures for reusable patch components may utilize gamma irradiation or ethylene oxide treatments that may eliminate microbial contamination. The sterilization protocols may be validated to ensure effectiveness while maintaining sensor functionality. The sterile packaging systems may protect sensors until clinical use.

The quality assurance protocols may include pre-treatment functionality checks that may verify sensor operation before patient application. The quality control procedures may test communication links, battery status, and measurement accuracy. The acceptance criteria may define performance thresholds that sensors must meet for clinical use.

The integration interfaces with existing treatment planning systems may utilize standardized communication protocols such as DICOM or HL7. The interface software may translate sensor data into formats compatible with treatment planning databases. The integration may enable automatic updating of patient records with real-time dosimetric measurements.

### Alternative Embodiments and Modifications

The disclosed systems and methods may be implemented in various alternative configurations without departing from the scope of the disclosure. Different sensor technologies may be utilized for radiation detection. Alternative communication protocols may be employed for data transmission between sensors and treatment planning systems.

Various external radiation therapy devices may be integrated with the disclosed systems. Different AI/ML algorithms may be implemented for treatment optimization. Alternative user interface designs may be provided for clinical operators.

The systems may be scaled for different clinical environments from large medical centers to smaller institutions. Various sensor configurations may be employed based on specific treatment requirements. Different data storage and analysis approaches may be implemented based on institutional needs.

The disclosed embodiments may represent only some of many possible implementations of the subject matter. Features from different embodiments may be combined in various ways. Additional features may be incorporated, and described features may be modified or omitted, all without departing from the scope of the disclosure.

While this specification contains many specifics, these may not be construed as limitations on the scope of what may be claimed, but rather as descriptions of particular implementations of the subject matter. Certain features that may be described in this specification in the context of separate embodiments may also be implemented in combination in a single embodiment. Conversely, various features that may be described in the context of a single embodiment may also be implemented in multiple embodiments separately or in any suitable subcombination.

Those of skill in the art may appreciate that the various illustrative blocks, modules, elements, components, methods, and algorithms described herein may be implemented as electronic hardware, computer software, or combinations of both. To illustrate this interchangeability of hardware and software, various illustrative blocks, modules, elements, components, methods, and algorithms may be described generally in terms of their functionality.

The subject technology may be illustrated according to various aspects described herein. The present disclosure may be provided to enable any person skilled in the art to practice the various aspects described herein. The disclosure may provide various examples of the subject technology, and the subject technology may not be limited to these examples.

A reference to an element in the singular may not be intended to mean “one and only one” unless specifically stated, but rather “one or more.” The term “some” may refer to one or more unless specifically stated otherwise. Pronouns in the masculine may include the feminine and neuter gender and vice versa.

The phrase “at least one of” preceding a series of items, with the terms “and” or “or” to separate any of the items, may modify the list as a whole, rather than each member of the list. The phrase “at least one of” may not require selection of at least one item; rather, the phrase may allow a meaning that includes at least one of any one of the items, and/or at least one of any combination of the items, and/or at least one of each of the items.

All structural and functional equivalents to the elements of the various configurations described throughout this disclosure that may be known or later come to be known to those of ordinary skill in the art may be expressly incorporated herein by reference and intended to be encompassed by the subject technology.

Generally, consistent with embodiments of the disclosure, program modules may include routines, programs, components, data structures, and other types of structures that may perform particular tasks or that may implement particular abstract data types. Moreover, embodiments of the disclosure may be practiced with other computer system configurations, including hand-held devices, multiprocessor systems, microprocessor-based or programmable consumer electronics, minicomputers, mainframe computers, and the like. Embodiments of the disclosure may also be practiced in distributed computing environments where tasks are performed by remote processing devices that are linked through a communications network. In a distributed computing environment, program modules may be located in both local and remote memory storage devices.

Furthermore, embodiments of the disclosure may be practiced in an electrical circuit comprising discrete electronic elements, packaged or integrated electronic chips containing logic gates, a circuit utilizing a microprocessor, or on a single chip containing electronic elements or microprocessors. Embodiments of the disclosure may also be practiced using other technologies capable of performing logical operations such as, for example, AND, OR, and NOT, including but not limited to mechanical, optical, fluidic, and quantum technologies. In addition, embodiments of the disclosure may be practiced within a general purpose computer or in any other circuits or systems.

Embodiments of the disclosure, for example, may be implemented as a computer process (method), a computing system, or as an article of manufacture, such as a computer program product or computer readable media. The computer program product may be a computer storage media readable by a computer system and encoding a computer program of instructions for executing a computer process. The computer program product may also be a propagated signal on a carrier readable by a computing system and encoding a computer program of instructions for executing a computer process. Accordingly, the present disclosure may be embodied in hardware and/or in software (including firmware, resident software, micro-code, etc.). In other words, embodiments of the present disclosure may take the form of a computer program product on a computer-usable or computer-readable storage medium having computer-usable or computer-readable program code embodied in the medium for use by or in connection with an instruction execution system. A computer-usable or computer-readable medium may be any medium that can contain, store, communicate, propagate, or transport the program for use by or in connection with the instruction execution system, apparatus, or device.

The computer-usable or computer-readable medium may be, for example but not limited to, an electronic, magnetic, optical, electromagnetic, infrared, or semiconductor system, apparatus, device, or propagation medium. More specific computer-readable medium examples (a non-exhaustive list), the computer-readable medium may include the following: an electrical connection having one or more wires, a portable computer diskette, a random access memory (RAM), a read-only memory (ROM), an erasable programmable read-only memory (EPROM or Flash memory), an optical fiber, and quantum computing elements. Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

Embodiments of the present disclosure, for example, are described above with reference to block diagrams and/or operational illustrations of methods, systems, and computer program products according to embodiments of the disclosure. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

While certain embodiments of the disclosure have been described, other embodiments may exist. Furthermore, although embodiments of the present disclosure have been described as being associated with data stored in memory and other storage mediums, data can also be stored on or read from other types of computer-readable media, such as secondary storage devices, like hard disks, solid state storage (e.g., USB drive), or a CD-ROM, a carrier wave from the Internet, or other forms of RAM or ROM. Further, the disclosed methods' stages may be modified in any manner, including by reordering stages and/or inserting or deleting stages, without departing from the disclosure.

All rights including copyrights in the code included herein are vested in and the property of the Applicant. The Applicant retains and reserves all rights in the code included herein, and grants permission to reproduce the material only in connection with reproduction of the granted patent and for no other purpose.

What is claimed is:

1. A method for dynamic treatment planning and realtime radiation awareness, comprising:
receiving a radiation treatment plan for a patient, or a radiation safety protocol for personnel or equipment operating in a radiation environment, the plan or protocol comprising a plurality of sessions or operational thresholds;
arranging a real time dose sensor proximate to a skin surface of the patient or integrated into a wearable patch, uniform, protective gear, vehicle, drone, tank, building, or other equipment platform used by soldiers, first responders, defense personnel, health security personnel, or radiation workers;
during a first session of the radiation treatment plan or during exposure to environmental radiation, providing to the patient using an external radiation therapy device a first prescribed radiation dose to a target volume of the patient, or detecting ambient radiation exposure in the field environment across personnel and equipment;
receiving from the sensor a measurement of actual radiation dose delivered to the target volume, absorbed by personnel, or accumulated by equipment;
modifying, based on the measurement of actual radiation, at least a second session of the radiation treatment plan or generating a real time alert, decision-support output, exposure log, or system-wide situational awareness update, resulting in a modified second session or enhanced operational awareness; and
during the modified second session of the radiation treatment plan, or during continued monitoring of the operational environment, providing to the patient using the external radiation therapy device a second prescribed radiation dose to a target volume of the patient or providing ongoing situational awareness of radiation exposure across personnel and equipment to safeguard operations.

2. The method of claim 1, wherein the external radiation therapy device is a gamma ray therapy device.

3. The method of claim 1, wherein the external radiation therapy device is a proton therapy device.

4. The method of claim 1, wherein the external radiation therapy device is a linear accelerator.

5. The method of claim 1, wherein modifying the second session or alert output comprises replacing an initial prescribed radiation dose with the second prescribed radiation dose, or generating an updated alert based on the detected exposure.

6. The method of claim 1, wherein modifying the second session or alert output comprises replacing an initial prescribed radiation duration with the second prescribed radiation duration, or issuing an immediate notification of excessive exposure.

7. The method of claim 1, wherein the real time dose sensor is a first real time dose sensor, the method further comprising arranging a second real time dose sensor proximate to the skin surface of the patient, on additional personnel, or embedded in additional equipment, wherein modifying the second session or generating the alert is further based on a measurement of the direction or source of the radiation, and wherein the measurement of the direction of the radiation is based on data from multiple sensors.

8. The method of claim 1, further comprising:
arranging a real time dose sensor proximate to a skin surface of a patient, on personnel, or within equipment, wherein the real time dose sensor comprises an electronic polymer dosimeter configured to detect radiation exposure through conductivity changes in pi-conjugated polymer materials;
during radiation exposure, receiving from the real time dose sensor measurements of actual radiation dose delivered to a target volume, accumulated by personnel, or measured across equipment; and
providing sub-milliliter spatial resolution for radiation dose monitoring in cancer therapy or realtime field awareness in operational environments.

9. The method of claim 1, further comprising:
positioning an electronic polymer dosimeter proximate to a radiation source, personnel, or equipment, wherein the electronic polymer dosimeter comprises pi-conjugated polymer materials configured to exhibit conductivity changes upon radiation exposure;
detecting radiation exposure through conductivity changes in the pi-conjugated polymer materials;
generating electrical signals corresponding to the conductivity changes; and
providing sub-milliliter spatial resolution for realtime radiation awareness during radiation detection in operational environments.

10. The method of claim 1, further comprising:
arranging a real time dose sensor proximate to a skin surface of a patient undergoing cancer therapy, or onto personnel or equipment operating in a radiation environment, wherein the real time dose sensor comprises an electronic polymer dosimeter configured to detect radiation exposure through conductivity changes in pi-conjugated polymer materials;
during delivery of therapeutic radiation or operational exposure, monitoring radiation dose through the electronic polymer dosimeter comprising pi-conjugated polymer materials configured to exhibit conductivity changes upon radiation exposure; and
providing sub-milliliter spatial resolution for precision cancer therapy or realtime field awareness in radiation safety operations.

1-20. (canceled)

21. A system for dynamic treatment planning and realtime radiation awareness, comprising:
a real time dose sensor configured to be arranged proximate to a skin surface of a patient or integrated into a wearable patch, uniform, protective gear, vehicle, drone, tank, building, or other equipment platform used by soldiers, first responders, defense personnel, health security personnel, or radiation workers, wherein the real time dose sensor comprises an electronic polymer dosimeter configured to detect radiation exposure through conductivity changes in pi conjugated polymer materials;
an external radiation therapy device configured to provide a first prescribed radiation dose to a target volume of the patient during a first session of a radiation treatment plan, or configured to operate in a radiation environment where ambient radiation exposure is detected across personnel and equipment;
a treatment planning engine configured to:
receive a radiation treatment plan for the patient comprising a plurality of sessions, or receive a radiation safety protocol for personnel or equipment operating in the radiation environment comprising operational thresholds;
receive from the real time dose sensor a measurement of actual radiation dose delivered to the target volume, absorbed by personnel, or accumulated by equipment;
modify, based on the measurement of actual radiation, at least a second session of the radiation treatment plan or generate a real time alert, decision-support output, exposure log, or system-wide situational awareness update, resulting in a modified second session or enhanced operational awareness; and
transmit control signals to the external radiation therapy device to provide a second prescribed radiation dose to the target volume of the patient during the modified second session, or provide ongoing situational awareness of radiation exposure across personnel and equipment to safeguard operations.

22. The system of claim 21, wherein the external radiation therapy device comprises a gamma ray therapy device.

23. The system of claim 21, wherein the external radiation therapy device comprises a proton therapy device.

24. The system of claim 21, wherein the external radiation therapy device comprises a linear accelerator.

25. The system of claim 21, wherein the treatment planning engine is configured to modify the second session or alert output by replacing an initial prescribed radiation dose with the second prescribed radiation dose, or by generating an updated alert based on the detected exposure.

26. A wearable dosimeter apparatus for dynamic treatment planning and realtime radiation awareness, comprising:
a flexible substrate configured to conform to a skin surface of a patient or to be integrated into a wearable patch, uniform, protective gear, vehicle, drone, tank, building, or other equipment platform used by soldiers, first responders, defense personnel, health security personnel, or radiation workers;
an electronic polymer dosimeter disposed on the flexible substrate and comprising pi-conjugated polymer materials configured to exhibit conductivity changes upon radiation exposure;
signal processing circuitry configured to:
detect the conductivity changes in the pi-conjugated polymer materials;
generate electrical signals corresponding to the conductivity changes; and
convert the electrical signals to digital dosimetric data representing actual radiation dose delivered to a target volume, absorbed by personnel, or accumulated by equipment; and
a communication module configured to transmit the digital dosimetric data to a treatment planning system or a radiation awareness platform.

27. The wearable dosimeter apparatus of claim 26, wherein the electronic polymer dosimeter comprises a three-dimensional embedded electrode structure configured to provide sub-milliliter spatial resolution for radiation dose monitoring.

28. The wearable dosimeter apparatus of claim 26, further comprising a power management circuit configured to:
monitor battery voltage and state-of-charge;
implement sleep modes during periods of inactivity; and
provide low-battery warnings.

29. The wearable dosimeter apparatus of claim 26, wherein the communication module is configured to transmit the digital dosimetric data wirelessly via at least one of Wi-Fi, Bluetooth, cellular communication, near-field communication, or radio frequency identification.

30. The wearable dosimeter apparatus of claim 26, further comprising:
a user interface comprising at least one of LED indicators, an OLED display, tactile switches, vibration motors, or audio speakers;
an alert generation circuit configured to activate the user interface when the digital dosimetric data exceeds a predetermined threshold;
environmental sensors configured to monitor at least one of temperature, humidity, pressure, acceleration, geographic location, or orientation; and
non-volatile memory configured to store calibration parameters, measurement data, and system status information.
